(19) United States
(12) Patent Application Publication
(10) Pub. No.: US 2004/0116936 A1
(43) Pub. Date: Jun. 17, 2004

(54) IOL loading guide

(76) Inventor: Randolph L. Seil, House Springs, MO (US)

Correspondence Address: Bausch & Lomb Incorporated, One Bausch & Lomb Place, Rochester, NY, 14604-2701, US

(21) Appl. No.: 10/318,478

(22) Filed: Dec. 12, 2002

(51) Int. Cl. A61F 9/00

(52) U.S. Cl. 606/107

(57) ABSTRACT

An IOL loading guide used in combination with an IOL cartridge for advancing the IOL deeper into the cartridge in a controlled, precise and predictable manner comprising a push rod having a rod tip and a sleeve surrounding the rod, the sleeve including a first longitudinally extending slot for engaging a portion of the cartridge as the cartridge is mounted thereon and thereby rotationally fixing the cartridge relative to the sleeve. As the cartridge is advanced onto the guide, the push rod engages and pushes the IOL deeper into the cartridge. The cartridge is then removed from the guide and used in combination with a handpiece having a plunger which engages and expels the IOL from the cartridge into the eye.

### BACKGROUND OF THE INVENTION

The present invention relates to surgical instruments, and more particularly relates to a manually operable surgical cataract instrument having an intraocular lens (hereinafter IOL) loading guide. Optionally, the instrument also includes a pair of forceps blades at the end opposite the IOL loading guide The IOL loading guide of the instrument is used to advance an IOL deeper into the lumen of a cartridge which is thereafter used in combination with a plunger device to advance the IOL completely through the cartridge and into the eye.

A common and desirable method of treating a cataract eye is to remove the clouded, natural lens and replace it with an artificial IOL in a surgical procedure known as cataract extraction. In the extracapsular extraction method, the natural lens is removed from the capsular bag while leaving the posterior part of the capsular bag (and preferably at least part of the anterior part of the capsular bag) in place within the eye. In this instance, the capsular bag remains anchored to the eye's ciliary body through the zonular fibers. In an alternate procedure known as intracapsular extraction, both the lens and capsular bag are removed in their entirety by severing the zonular fibers and replaced with an IOL which must be anchored within the eye absent the capsular bag. The intracapsular extraction method is considered less attractive as compared to the extracapsular extraction method since in the extracapsular method, the capsular bag remains attached to the eye's ciliary body and thus provides a natural centering and locating means for the IOL within the eye. The capsular bag also continues its function of providing a natural barrier between the aqueous humor at the front of the eye and the vitreous humor at the rear of the eye. IOLs are sometimes also implanted within an eye where the natural lens remains intact (phakic eye).

In each of the above-described surgical procedures, the surgeon cuts an incision into the cornea wherethrough the IOL is passed and implanted within the eye. Various instruments and methods for implanting the IOL in the eye are known. In one method, the surgeon simply uses surgical forceps having opposing blades which are used to grasp the IOL and insert it through the incision into the eye. While this method is still practiced today, more and more surgeons are using more sophisticated IOL delivery devices which offer advantages such as affording the surgeon more control when inserting the IOL into the eye. IOL delivery devices have recently been developed with reduced diameter insertion tips which allow for a much smaller incision to be made in the cornea than is possible using forceps alone. Smaller incision sizes (e.g., less than about 3 mm) are preferred over larger incisions (e.g., about 3.2 to 5+ mm) since smaller incisions have been attributed to reduced post-surgical healing time and complications such as induced astigmatism.

Since IOLs are very small and delicate articles of manufacture, great care must be taken in their handling. In order for the IOL to fit through the smaller incisions, they need to be folded and/or compressed prior to entering the eye wherein they will assume their original unfolded/uncompressed shape. The IOL delivery devices must therefore be designed in such a way as to permit the easy passage of the IOL through the device and into the eye, yet at the same time not damage the delicate IOL in any way. Should the IOL be damaged during delivery into the eye, the surgeon will most likely need to extract the damaged IOL from the eye and replace it with a new IOL, a highly undesirable surgical outcome.

Thus, as explained above, the IOL delivery device must be designed to permit easy passage of the IOL therethrough. It is equally important that the IOL be expelled from the tip of the delivery device and into the eye in a predictable orientation and manner. Should the IOL be expelled from the tip in the wrong orientation, the surgeon must manipulate the IOL in the eye which could result in trauma to the surrounding tissues of the eye. It is therefore highly desirable to have a delivery device which will expel the IOL from the delivery device tip and into the eye in a controlled, predictable and repeatable manner.

To ensure controlled expression of the IOL through the tip of the delivery device, the IOL must first be loaded into the IOL delivery device. The loading of the IOL into the delivery device is therefore a precise and very important step in the process. Incorrect loading of an IOL into the delivery device is oftentimes cited as the reason for a failed IOL delivery sequence. The interface between the IOL and the delivery device also is a critical parameter of the IOL delivery sequence. Various delivery devices have been proposed which attempt to address the problem of IOL loading into the delivery device, yet there remains a need for an improved method for loading an IOL into the lumen of a delivery device.

### SUMMARY OF THE INVENTION

The present invention is a surgical instrument used in cataract surgery wherein an IOL is implanted into a patient's eye using an IOL delivery device having a cartridge component wherein the IOL is loaded prior to insertion thereof into the eye. The inventive surgical instrument comprises an IOL loading guide having a push rod with a rod tip lying along a first axis, and a sleeve coaxially surrounding the push rod and having an open end adjacent the rod tip, the sleeve including a first longitudinally extending opening defining a keyed slot such that the cartridge may be mounted to the loading guide in only a specific rotational orientation. The IOL is first preloaded into the cartridge whereupon the IOL assumes a folded position adjacent the proximal end of the cartridge. The IOL may be advanced deeper into the cartridge passageway by mounting the cartridge onto the IOL loading guide of the invention and sliding the cartridge therealong until the cartridge abuts a stop located at the end of the keyed slot opposite the rod tip. As the cartridge is mounted on the guide, the rod tip engages and advances the IOL deeper into and toward the distal end of the cartridge as the cartridge is fully advanced onto said IOL loading guide. Once the cartridge has been fully advanced on the guide, the cartridge is removed from the IOL loading guide with the IOL positioned adjacent the distal end of the cartridge in a known orientation.

In a second yet optional aspect of the invention, the surgical instrument includes forceps having a pair of blades located at the end of the instrument opposite the cartridge guide. The blades are used to manipulate and preload the IOL into the cartridge.

In yet a further aspect of the invention, a method of loading an IOL into an IOL cartridge having opposite proximal and distal ends and a longitudinal passageway extending therebetween is disclosed, the method comprising the steps of:

a) preloading the IOL into the cartridge passageway with the IOL coming to rest adjacent the proximal end of the cartridge;

b) mounting the cartridge onto an IOL loading guide having a rod with opposite proximal and distal ends and a sleeve surrounding the rod, the sleeve being configured for telescoping, mating engagement with the cartridge, the rod distal end engaging and advancing the IOL deeper into the cartridge passageway as the cartridge is moved along the sleeve toward the proximal end of the rod whereupon the IOL comes to rest at a location closer to the distal end of the cartridge;

c) removing the cartridge from the IOL loading guide;

d) advancing a plunger through the cartridge from the proximal to the distal end thereof whereby the plunger pushes the IOL completely through the cartridge and into a patient's eye.

### BRIEF DESCRIPTION OF THE DRAWING

FIG. 1 is a perspective view of a prior art surgical instrument;

FIG. 2a is a perspective view of an IOL cartridge in the open position;

FIG. 2b is a perspective view of the IOL cartridge taken from the opposite perspective of FIG. 2a;

FIG. 2c is a side elevational view of the cartridge of FIGS. 2a and 2b;

FIG. 3 is a plan view of an IOL;

FIG. 4a is a side elevational view of the inventive surgical instrument;

FIG. 4b is the view of FIG. 4a with the instrument rotated about its longitudinal axis approximately 90°;

FIG. 5A is a perspective view of the inventive surgical instrument showing the cartridge in spaced relation to the IOL loading guide;

FIG. 5B is the view of FIG. 5A shown with the IOL cartridge loaded onto the IOL loading guide thereof,

FIG. 5c is a Cross-sectional view of the sleeve portion of the IOL loading guide as taken generally along the line 5c-5c of FIG. 5A;

FIG. 5d is an end view of the cartridge;

FIG. 6A is the view of FIG. 5A showing the cartridge in spaced relation to the IOL loading guide;

FIG. 6B is the view of FIG. 6A shown with the IOL cartridge loaded onto the IOL loading guide;

FIG. 7a is a side elevational view of the IOL cartridge showing the IOL loaded therein in an initially loaded position adjacent the proximal end of the cartridge;

FIG. 7b is the view of FIG. 7a with the IOL shown in an advanced position adjacent the tip portion of the cartridge subsequent to the IOL cartridge having been mounted and then withdrawn from the IOL loading guide of the surgical instrument; and

FIG. 8 is a perspective view showing the cartridge, the inventive surgical instrument and an insertion handpiece which are used in conjunction with each other to deliver an IOL into a patient's eye.

### DETAILED DESCRIPTION OF PREFERRED EMBODIMENT

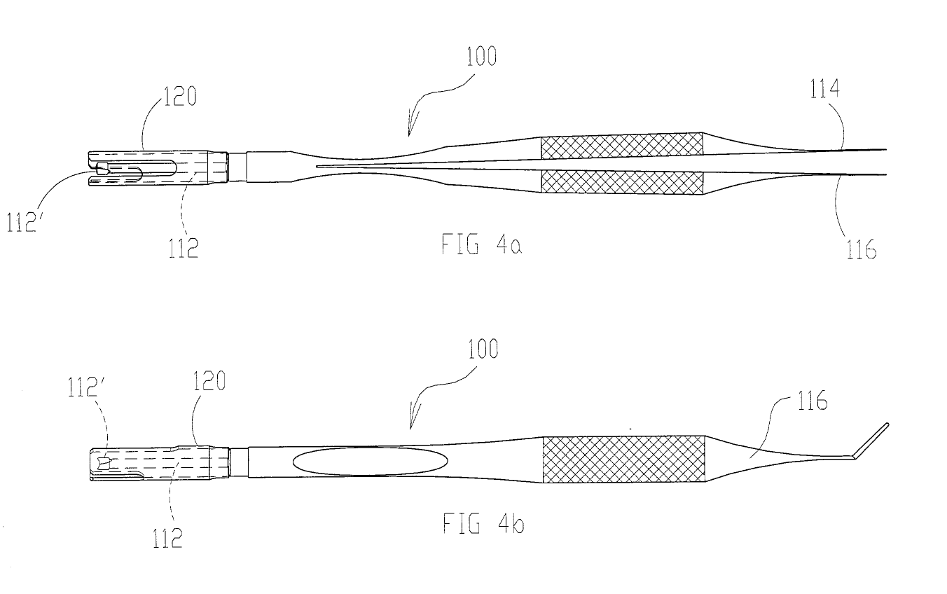

In an eye where the natural crystalline lens has been damaged (e.g., clouded by cataracts), the natural lens is no longer able to properly focus and direct incoming light to the retina and images become blurred. A well known surgical technique to remedy this situation involves removal of the damaged crystalline lens which may be replaced with an artificial lens known as an intraocular lens (hereinafter IOL) such as prior art IOL 20 seen in FIG. 3.

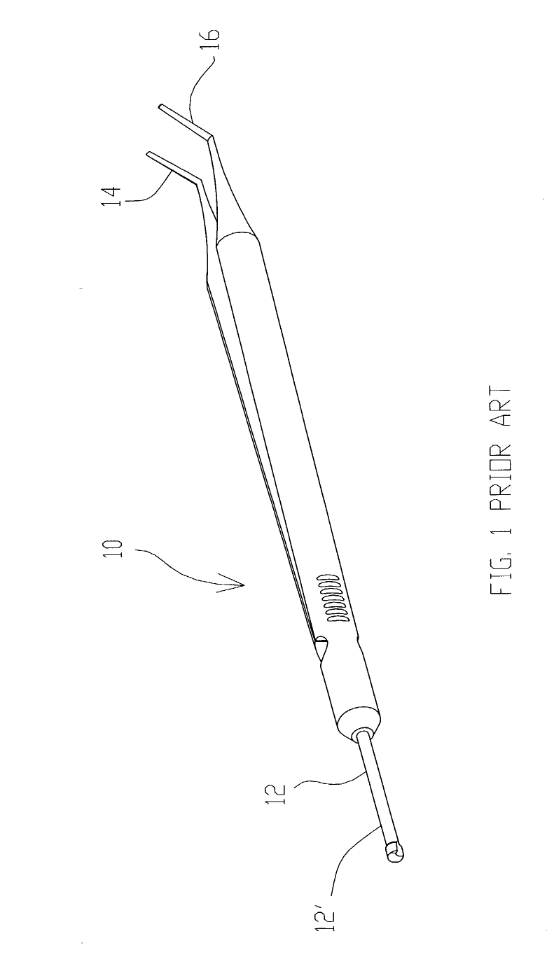

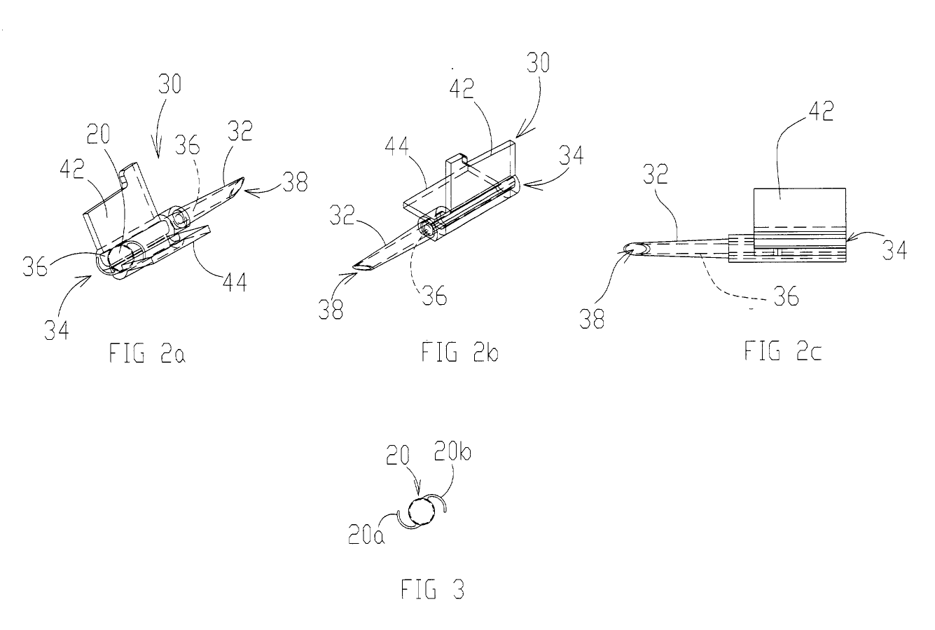

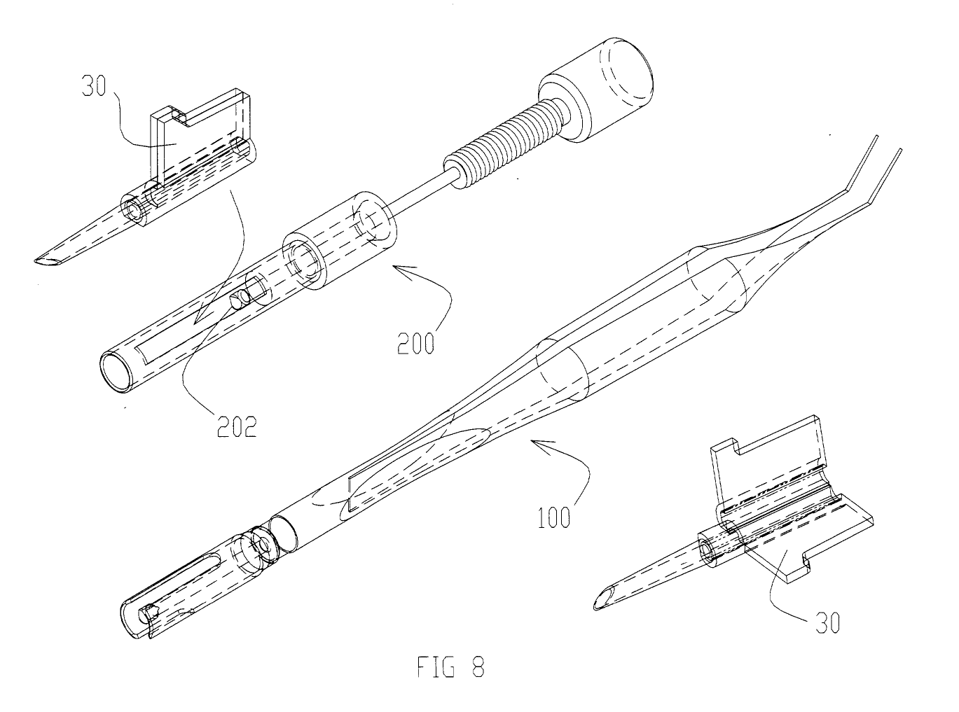

A prior art surgical instrument 10 is seen in FIG. 1 and includes push rod 12 at one end and a pair of opposing blades 14 and 16 at the opposite end thereof. Instrument 10 is used in cataract surgeries to assist in the implantation of an IOL into an eye. More particularly, instrument 10 is used to facilitate loading of an IOL 20 (FIG. 3) into an IOL cartridge, such as winged cartridge 30 seen in FIGS. 2a-c. Cartridge 30 is of the so-called “wing” type used to fold IOL 20 so that it may be passed through the cartridge tip 32 and expressed into the eye. This is accomplished by mounting the cartridge 30 and IOL 20 therein onto a separate insertion handpiece 200 (see FIG. 8) which has a plunger 202 for engaging IOL 20 from the proximal open end 34 of the cartridge. With the cartridge tip inserted through the incision made in the eye, the handpiece plunger 202 is advanced to push the IOL 20 through the cartridge lumen 36 in the direction of open distal end 38 wherethrough IOL 20 is expressed from the cartridge tip 32 and into the eye.

As mentioned above, instrument 10 is used to assist in loading the IOL 20 into the cartridge 30. In a first step, the doctor or assistant begins by grasping IOL 20 with blades 14,16 which operate as forceps, and places IOL 20 into the open cartridge 30 as seen in FIG. 2a. The tips of blades 14,16 may also be used to press the IOL 20 downwardly against lumen 36 just prior to closing cartridge 30 by bringing wings 42,44 together. In this way, flexible IOL 20 becomes folded within lumen 36 adjacent the proximal end 34 of cartridge 30.

The positioning of IOL 20 within cartridge 30 is directly related to the manner in which it will move as it is pushed through lumen 36 and expressed from tip 38 into the eye. For example, should IOL 20 not freely and easily be pushed by the advancing plunger 202 of the handpiece 200, IOL 20 could be easily damaged due to its fragile nature. If IOL 20 is damaged during the delivery process, it may have to be removed from the eye and replaced with a new IOL. Another important factor to consider is how the IOL will move as it is expressed from tip 38 into the eye. As the IOL 20 is expressed from tip 38, it resumes its original, unfolded shape. If the unfolding action of IOL 20 is not controlled, unexpected contact may occur between the IOL and surrounding eye tissues resulting in a possible trauma to the eye. In addition, the IOL may require in-vivo manipulation to correct the position thereof in the eye. In-vivo IOL manipulation should be minimized to reduce the chance of damage to the IOL and/or surrounding tissue. It is therefore very desirable to deliver an IOL into the eye in a controlled manner.

Referring still to FIGS. 1, 2a-c and 3, the IOL 20 may be further advanced within lumen 36 by using push rod 12 on the end of instrument 10 opposite blades 12,14. More particularly, the doctor or assistant inserts the tip 12′ of rod 12 into open proximal end 34 wherupon tip 12′ engages IOL 20 located therein as previously described. Rod 12 is advanced further into lumen 36 until IOL 20 is positioned further toward tip 32. At this point, rod 12 is withdrawn from lumen 36 and the cartridge 30 with IOL 20 positioned therein is placed in combination with an insertion handpiece such as handpiece 200 having a plunger 202 which is selectively advanced by the surgeon through lumen 36. Thus, with the tip 32 inserted into the eye, the surgeon advances the plunger to engage and expel IOL 20 from tip 38 into the eye.

It will be appreciated from the foregoing that while rod 12 of prior art instrument 10 is useful for advancing IOL 20 deeper into the cartridge lumen 36, the engagement profile between rod 12 and the IOL 20 is not controlled in either a linear or rotational sense. That is, rod 12 may be inserted into cartridge proximal end 34 at any arbitrary rotational angle, and/or it may be offset from the longitudinal axis of lumen 36, and/or it may be inserted at any arbitrary depth into the cartridge lumen 36. This arbitrary insertion profile adds uncertainty as to how the rod tip 12′ will engage the IOL 20. As explained above, uncontrolled engagement profiles between the IOL and advancement tips creates the chance for damage to occur to the IOL and/or an uncontrolled IOL delivery profile, both of which are unwanted occurrences.

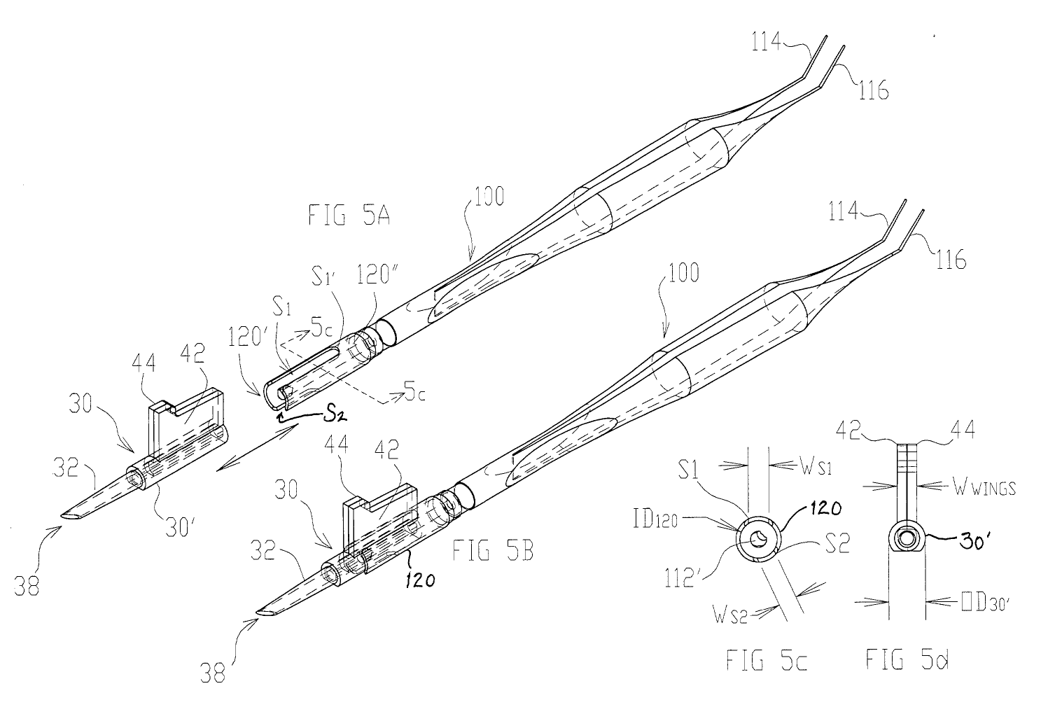

Referring to FIGS. 4-7, improved surgical instrument 100 is seen to include an IOL loading guide having a keyed sleeve 120 which surrounds push rod 112 having a rod tip 112′. Keyed sleeve 120 extends in generally coaxial and spaced relationship to rod 12. Keyed sleeve 120 is configured for mating, removable engagement with cartridge 30 and operates to advance IOL 20 deeper into the cartridge while maintaining linear advancement control as well as rotational and axial alignment between push rod 112 and cartridge 30. As such, the engagement profile between rod tip 12′ and IOL 20, and thus also the positioning of IOL 20 within lumen 36, is predictable, controlled and consistent. Instrument 100 may consist of the IOL loading guide alone, or may optionally include at the opposite end thereof a pair of opposable blades 114,116 used to grasp and manipulate an IOL 20 for loading into cartridge 30 in generally the same manner as described with reference to blades 14,16 of instrument 10. Of course other types and configurations of IOL surgical instrumentation (or none at all) may be substituted for forceps blades 114,116 on instrument 100, as desired.

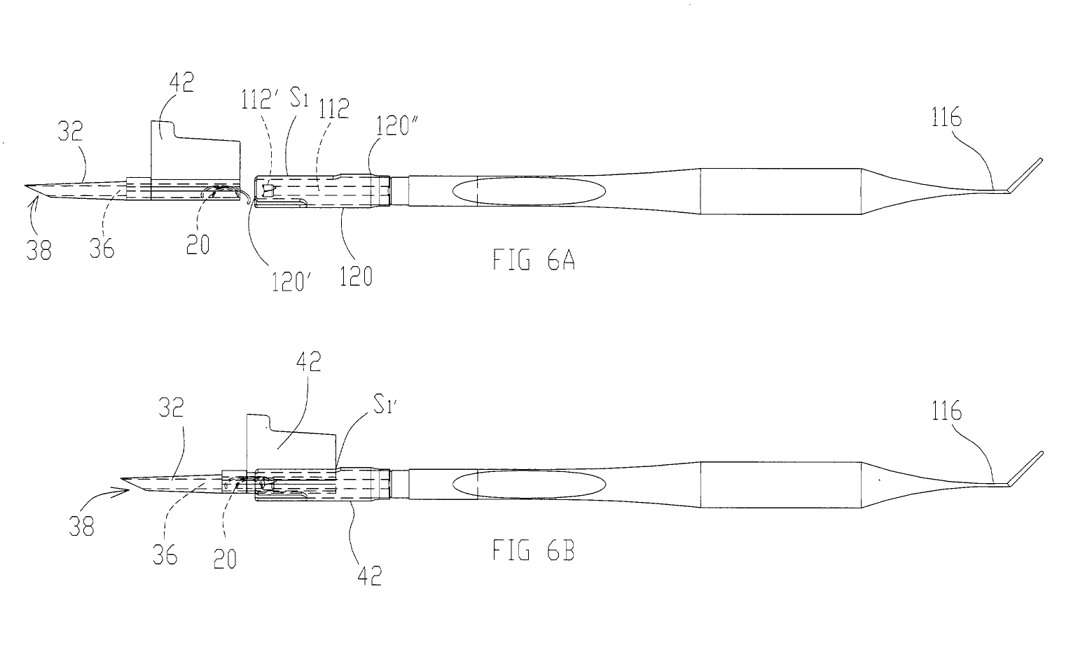

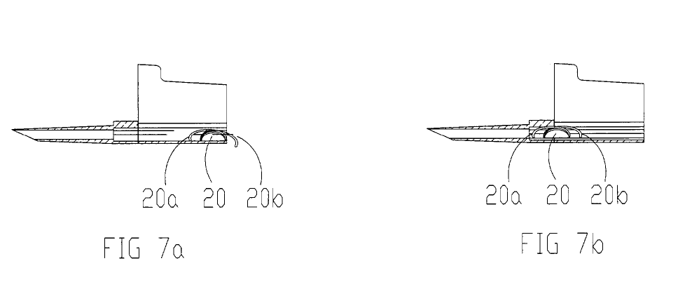

More particularly and as seen in FIGS. 5a-d, sleeve 120 of the IOL loading guide is of generally cylindrical configuration to match the generally cylindrical shape of the closed cartridge body 30′, and includes an inner diameter ID₁₂₀ slightly larger than the outer diameter OD_{30′} of the cartridge body 30′. As such, cartridge body 30′ may be telescopingly engaged within sleeve 120 as seen in FIG. 5B and 6B. A first slot S₁ is formed in sleeve 120 extending from the open, distal end 120′ thereof toward proximal end 120″ thereof Slot S₁ terminates, forming a stop S₁′ at a location adjacent the sleeve proximal end 120″. Slot S₁ is configured and sized to allow clearance for closed wings 42,44 of cartridge 30 as cartridge 30 is engaged with sleeve 120. It will be appreciated that since wings 42,44 must engage slot S₁ in order for cartridge 30 to be fully advanced onto sleeve 120, cartridge 30 can only be mounted onto sleeve 120 in a specific rotational orientation. This feature thus adds control to the engagement profile between push rod tip 112′ and IOL 20 within cartridge 30.

As previously described, the surgeon or assistant begins the operation by loading IOL 20 into cartridge 30 by placing IOL 20 into the open cartridge (with wings 42,44 splayed open as seen in FIG. 2a). Forceps 114,116 may be used to grasp and place IOL 20 into the open cartridge with the IOL haptics 20a,b facing toward the proximal and distal ends 34,32 of cartridge 30, respectively. In this regard, it is noted the IOL packaging typically includes directions for use which describe and illustrate proper IOL orientation and placement in the open cartridge 30. With the IOL 20 so positioned, wings 42,44 are brought together which causes IOL 20 to fold upon itself, coming to rest inside cartridge lumen 36 in the same location where it was placed with the forceps, namely, adjacent cartridge proximal end 34. At this point, the surgeon or assistant mounts cartridge 30 to sleeve 120 by aligning wings 42,44 with slot S₁ and sliding cartridge 30 in the direction of sleeve proximal end 120″ until the wings abut stop S₁′. As this is done, push rod tip 112′ engages IOL 20 and advances (pushes) IOL 20 through cartridge lumen 36 until the cartridge comes to a stop against stop S₁′. At this point, IOL 20 has been advanced deeper into the cartridge lumen 36 to the position seen in FIGS. 6B and 7B. It will be appreciated that the lengths of the push rod 112 and slot 120 may be either increased or decreased relative to the length of cartridge 30 to dictate exactly where in the lumen 36 the IOL 30 will come to rest. Thus, if it is desired to have the IOL advanced to a position more closely adjacent tip open end 38, sleeve 12 and slot 120 may be lengthened, and vice versa.

In this regard, the main consideration as to exactly where IOL 20 should come to rest within cartridge 30 is to maximize, to the extent possible, the control of the expression sequence of IOL 20 out of cartridge 30 and into the eye. By “expression sequence” it is meant the IOL delivery parameters such as expression rate and IOL orientation as it exits the tip and unfolds within the eye, for example. These factors will necessarily vary depending on the cartridge and IOL design, dimensions and materials, and thus the exact location where IOL 20 should come to rest in the cartridge using instrument 100 will likewise vary; however, this should be readily determinable through basic testing with the particular cartridge and IOL being used by those skilled in the art.

It is quite important the haptics 20a, 20b of the IOL do not get caught up on anything as the IOL is placed into and then advanced through the cartridge. User instructions therefore typically instruct the user to initially load the IOL into the cartridge with the leading haptic pointing toward the distal tip 32 and the trailing haptic pointing to the proximal end 34 of the cartridge lumen. The tip of the plunger components (i.e., 112′ on instrument 100 and 202 on insertion handpiece 200) are also configured with a notch or the like to provide clearance for the trailing haptic as the plunger is advanced into the cartridge, past the trailing haptic to engage the optic portion of the IOL. In some commercial IOL applications, the IOL loading instructions indicate that the IOL should be initially loaded into the cartridge with the trailing haptic dangling out the proximal end 34 of the cartridge. This is illustrated in the drawing in FIGS. 2A, 6A and 7A. In order to provide clearance for this trailing haptic as the cartridge is mounted onto the IOL guide of instrument 100, a second slot S₂ is formed in sleeve 120 at a location rotationally offset from first slot S₁. The degree to which the second slot S₂ is off-set from the first slot S₁ should match where the trailing haptic exits the proximal end of the lumen. Again, the user instructions tell the user where the trailing haptic should come to rest outside the lumen when initially loading an IOL within the cartridge. Of course in those applications where the trailing haptic is not required to initially dangle from the proximal end of the lumen, the second slot S₂ would not be necessary.

Second slot S₂ is not as wide as first slot S₁ or wings 42,44 so that wings 42,44 cannot be inadvertently inserted into second slot S₂, thereby maintaining the specified rotational orientation of cartridge 30 on sleeve 120 as described previously. Once fully advanced onto sleeve 120, cartridge 30 is simply withdrawn therefrom by sliding it in the opposite direction whereby rod tip 112′ disengages from IOL 20 and withdraws from cartridge 30. IOL 20 has thus been advanced deeper into the cartridge lumen 36 to a position closer to tip 32 as seen in FIG. 7A. At this position, the trailing haptic is now entirely within the lumen 36. While carefully maintaining wings 42,44 in the closed position with the thumb and finger, the surgeon or assistant loads cartridge 30 onto a separate insertion handpiece such as handpiece 200 seen in FIG. 8. Handpiece 200 includes a plunger 202 which the surgeon advances to enter the proximal end of cartridge 30 and push IOL 20 completely through cartridge 30 and into the eye.

While the invention has been described with reference to particular embodiments thereof including associated components, it will be understood that variations may be made without departing from the full spirit and scope of the invention as defined in the claims which follow. For example and as noted above, the configurations of the IOL, the cartridge and the handpiece may all differ from that described herein. The cartridge may by of any type, including those that do not open along their longitudinal axis and instead have the IOL initially loaded therein from the open, proximal end thereof Regarding the handpiece to which the cartridge is loaded for the IOL insertion into the eye, the plunger mechanism may vary from a screw-type as shown herein, to a push or syringe-type, for example. These and other modifications will be evident to those skilled in the art.

What is claimed is:

1. A surgical instrument for use in cataract surgery involving implantation of an IOL into a patient's eye including the use of an IOL cartridge having proximal and distal ends and a lumen extending therebetween and wherein an IOL is preloaded prior to insertion thereof into the eye, said surgical instrument including an IOL loading guide comprising:
i) a rod with a rod tip lying along a first axis;
ii) a sleeve having distal and proximal ends including a longitudinal passageway extending therebetween and through which said rod extends with said rod tip lying adjacent said sleeve distal end;
whereby the preloaded IOL may be advanced deeper into the cartridge lumen toward said distal end thereof by mounting the cartridge onto the IOL loading guide and sliding the cartridge toward the proximal end of the sleeve, said rod tip engaging and advancing the IOL deeper into said cartridge as said cartridge is slid onto said IOL loading guide.

2. The surgical instrument according to claim 1, said sleeve further including a first longitudinally extending slot and a cartridge stop defined at the termination of said first slot located adjacent said sleeve proximal end, said cartridge including one or more wing portions which engage said first slot as said cartridge is mounted onto said IOL loading guide such that said cartridge is rotationally fixed relative to said sleeve.

3. The surgical instrument according to claim 1, and further comprising a forceps having first and second forceps blades at an end of said instrument opposite said IOL loading guide.

4. The surgical instrument according to claim 2, and further comprising a second slot longitudinally extending along said sleeve, said second slot forming an opening through which an IOL haptic may pass as said cartridge is mounted upon said IOL loading guide.

5. The surgical instrument according to claim 1, wherein said rod and said sleeve are coaxial.

6. The surgical instrument according to claim 1, wherein said rod tip includes a notch.

7. The surgical instrument according to claim 1, and further comprising a forceps having opposing blades located at an end of said instrument opposite said IOL loading guide.

8. A method of loading an IOL into an IOL cartridge having opposite proximal and distal ends and a lumen extending therebetween, said method comprising the steps of:
a) preloading the IOL into the cartridge lumen with the IOL coming to rest adjacent the proximal end of the cartridge;
b) mounting the cartridge onto an IOL loading guide having a rod with opposite proximal and distal ends and a sleeve surrounding the rod, the sleeve being configured for telescoping, mating engagement with the cartridge, the rod distal end engaging and advancing the IOL deeper into the cartridge passageway as the cartridge is moved along the sleeve whereupon the IOL comes to rest at a location closer to the distal end of the cartridge; and
c) removing the cartridge from the IOL loading guide.

9. The method according to claim 8 wherein said IOL is preloaded into said cartridge lumen using a forceps having opposing blades located at an end of said instrument opposite said IOL loading guide.
